(19) United States
(12) Patent Application Publication
(10) Pub. No.: US 2022/0125818 A1
(43) Pub. Date: Apr. 28, 2022

(54) COMPOSITIONS AND METHODS FOR TREATMENT OF A CORONAVIRUS DISEASE (COVID-19) INFECTION

(71) Applicant: Emile Shenouda, South Gate, CA (US)

(72) Inventor: Emile Shenouda, South Gate, CA (US)

(21) Appl. No.: 17/089,344

(22) Filed: Nov. 4, 2020

Related U.S. Application Data
(60) Provisional application No. 63/105,864, filed on Oct. 26, 2020.

(51) Int. Cl. A61K 31/7052; A61K 31/65; A61K 31/675; A61K 31/4706; A61K 31/616; A61K 31/5377; A61K 31/4743; A61K 31/727

(52) U.S. Cl. CPC A61K 31/7052; A61K 31/65; A61K 31/675; A61K 31/4706; A61K 45/06; A61K 31/5377; A61K 31/4743; A61K 31/727; A61K 31/616

(57) ABSTRACT

Compositions and methods for treating COVID-19 infections are provided herein. The methods include administering a therapeutically effective amount of a combination of at least three components. The first component is selected from a group consisting of azithromycin, doxycycline, and remdesivir, the second component is selected from a group consisting of hydroxychloroquine and a corticosteroid, and the third component is selected from a group consisting of acetylsalicylic acid, rivaroxaban, clopidogrel, and enoxaparin sodium.

### CROSS-REFERENCE TO RELATED APPLICATIONS

The present application claims the benefit of priority to U.S. Provisional Patent Application No. 63/105,864, filed Oct. 26, 2020, which is hereby incorporated by reference in its entirety and is considered a part of this specification.

### FIELD OF THE INVENTION

This patent document relates generally to compositions and methods for treatment of Coronavirus disease 2019 (COVID-19). More particularly, the subject matter of this patent document relates to novel protocols for treatment of COVID-19.

### DESCRIPTION OF THE RELATED ART

COVID-19 infection is caused by a virus known as Severe Acute Respiratory Syndrome Coronavirus 2 (SARS-CoV-2). Older adults and people who have severe underlying medical conditions, like heart, lung disease or diabetes, appear to be at higher risk for developing more serious complications from COVID-19 illness. Since its initial discovery, it has spread throughout the world. As of Oct. 12, 2020, there have been over 37.5 million reported cases with more than 1.07 million deaths.

Transmission of the COVID-19 can be airborne and or by contact to thin mucus membranes that the virus can penetrate, for example, the eyes and the throat mucosa. Coronaviruses work by latching onto cells of another organism to cause a cascade effect that releases spike-shaped proteins on the virus' surfaces that contain the virus' genetic information into the cell. What differentiates coronaviruses is the RNA pseudoknot which stops transcription and moves the ribosomal RNA back one nucleotide. Exhibiting similar symptoms to the flu and pneumonia, its ability to mutate quickly, its resistance to antibodies—these are just a few of the factors that make it hard to cure COVID-19.

SARS-CoV-2 infection activates innate and adaptive immune response, thus, sustaining the resolution of COVID-19. While a rapid and well-coordinated immune response represents the first line of defense against viral infection, excessive inflammatory innate response and dysregulated adaptive host immune defense may cause harmful tissue damage at both at the site of virus entry and at systemic level. The excessive pro-inflammatory host response has been hypothesized to induce an immune pathology resulting in the rapid course of acute lung injury (ALI) and acute respiratory distress syndrome (ARDS) occurring in SARS-CoV-2 infected patients. The massive cytokine and chemokine release, the so-called “cytokine storm,” clearly reflects a widespread uncontrolled dysregulation of host immune defense.

Although several treatment options have been used, considered or experimented since the outbreak of the virus, none has been effective at treating severe cases or preventing the transition from mild to severe stages. Further, patients that recovered from COVID-19 can get reinfected depending on the antibodies formed against the virus strain from the previous infection. As such, vaccinations may not be a permanent protective tool. Thus, there is a need for an effective treatment protocol for COVID-19.

The present disclosure provides a treatment protocol for effective clinical management of severe cases of COVID-19, as well as for preventing the transition from mild to severe stages.

### SUMMARY

The present disclosure provides methods for treating COVID-19 infections, methods for reducing viral replication of SARS-CoV-2, methods for controlling the dysregulation of host immune defense, and/or methods for preventing coagulation and blood clot formation in COVID-19 infected patients. In one embodiment, the methods include administering a therapeutically effective amount of a combination of at least three components. The first component is selected from a group consisting of azithromycin, doxycycline, and remdesivir, the second component is selected from a group consisting of hydroxychloroquine and a corticosteroid, and the third component is selected from a group consisting of acetylsalicylic acid, rivaroxaban, clopidogrel, and enoxaparin sodium. In one embodiment, the methods further include administering a therapeutically effective amount of Vitamin C, Vitamin D3 and Zinc.

In one embodiment, the therapeutically effective amount of azithromycin may be a dosage of about 250 to 500 mg/day for a period of about 5 to 7 days, the therapeutically effective amount of doxycycline may be a dosage of about 100 mg every 12 hours for about 5 days, and the therapeutically effective amount of remdesivir may be a dosage administered intravenously comprising about 200 mg on the first day, followed by about 100 mg for days 2 to 5.

In another embodiment, the therapeutically effective amount of hydroxychloroquine may be a dosage of about 200 to about 400 mg/day for a period of about 5 to 7 days. As can be appreciated, corticosteroid may be prednisone, solumedrol, hydrocortisone and dexamethasone. The therapeutically effective amount of prednisone may be an oral dosage of about 20 mg-80 mg in a tapering dose over a period of about 7 days, the therapeutically effective amount of hydrocortisone may be an oral dosage of about 50 mg every 8 hours, and the therapeutically effective amount of dexamethasone may be a dosage of about 6 mg daily administered intravenously.

In yet another embodiment, the therapeutically effective amount of acetylsalicylic acid may be a dosage of about 81 mg daily for a period of about 30 days, the therapeutically effective amount of rivaroxaban may be a dosage of about 20 mg/day for a period of about 30 day, the therapeutically effective amount of clopidogrel may be a dosage of about 75 mg/day for about 30 days, and the therapeutically effective amount of enoxaparin sodium may be a dosage of about 40 mg SQ/day for about 14 days.

Each of the foregoing various aspects, together with those set forth in the claims and described in connection with the embodiments summarized above and disclosed herein may be combined to form claims for a composition, methods of manufacture and/or use in any way disclosed herein without limitation.

### DETAILED DESCRIPTION

Unique and inventive methods for the treatment of COVID-19 are disclosed herein. The methods are based on combination therapies to address at least three parameters: (1) slowing down the COVID-19 virus replication, (2) controlling the dysregulation of host immune defense, and (3) preventing coagulation and blood clot formation (which plays a major role in increasing the mortality in COVID-19 patient).

Slowing Down the Virus Replication

Viral mRNA can efficiently hijack host ribosomes during infection for translation of proteins necessary for virus propagation. Initiation of translation is a key step, during which the coding region of mRNA, open reading frame, gets properly positioned on the ribosome. Cryo-EM (electron cryo-microscopy) structures of the ribosome-bound Taura syndrome virus (TSV) messenger RNA (red) reveal a structural mechanism of initiation on viral mRNAs.

As can be appreciated, a first component of the combination therapy is directed to the slowing down of the virus replication. This may include administering a therapeutically effective amount of one or more of the following prescription drugs: Azithromycin (Zithromax®), Doxycycline, Remdesivir (Veklury®), generic formulations thereof or known substitutes thereof.

Azithromycin stops protein synthesis by inhibiting the transpeptidation/translocation step of protein synthesis and by inhibiting the assembly of the 50S ribosomal subunit. The antiviral effect of Azithromycin supports a large-spectrum antiviral activity. Azithromycin appears to decrease the virus entry into the cells. In addition, it may enhance the immune response against viruses by several actions. For example, Azithromycin up-regulates the production of type I and III interferons (including interferon-β and interferon-λ), as well as genes involved in virus recognition such as MDA5 and RIG-I. Azithromycin may decrease IL-8 levels. These mechanisms are universally involved in the innate response against infectious agents, and it appears to be effective against SARS-CoV-2. As can be appreciated, Azithromycin provides an immunomodulatory profile by inhibiting several cytokines involved in COVID-19 severe respiratory syndrome. It may further regulate and/or decrease the production of IL-1β, IL-6, IL-8, IL-10, IL-12, and IFN-α.

In one embodiment of the present disclosure, a therapeutically effective amount of Azithromycin (Zithromax®) for slowing down the virus replication of SARS-CoV-2 is a dosage of about 250 to 500 mg/day once a day for a period of about 5 to 7 days that is administered orally or intravenously.

Doxycycline and other tetracycline derivatives, such as minocycline, exhibit anti-inflammatory effects against several RNA viruses. The administration of these drugs have been associated with clinical improvement, including a reversal of cytokine storm in some infections caused by RNA viruses. Tetracycline derivatives may also provide transcriptional up-regulation of intracellular zinc finger antiviral protein (ZAP), which is an encoding gene in host cells. ZAP may bind to specific target viral mRNAs and represses their translation. Experimental administration of tetracycline has shown to induce the overexpression of host ZAP in HEK293, rats and monkeys cell lines (Vero cells), which contributed to inhibition of RNA viruses (i.e. Dengue, Ebola, Human Immunodeficiency Virus, Zika, and Influenza A viruses). Moreover, tetracycline derivatives such as doxycycline are highly lipophilic antimicrobials that chelate zinc compounds on matrix metalloproteinases (MMPs) of mammalian cells, and an in vitro experimentation have shown that murine coronaviruses rely on MMPs for cell fusion and viral replication. Doxycycline may also be used to target viral fusion and replication by coronaviruses utilizing host proteases. In the context of COVID-19, elevated levels of blood interleukin (IL)-6 have been more commonly observed in severe cases and among those that have died from the infection, which demonstrates that mortality may be due to a virally-driven hyperinflammation and a cytokine storm. This intense pro-inflammatory state is similar to the reactions experienced with dengue and hemorrhagic fever. Notably, doxycycline reduced pro-inflammatory cytokines in infected patients with dengue and hemorrhagic fever, reducing the mortality rate by about 46%, and was more effective than tetracycline in the reduction of these pro-inflammatory cytokines.

In an embodiment of the present disclosure, a therapeutically effective amount of Doxycycline for slowing down the virus replication of SARS-CoV-2 is a dosage of about 50 mg to about 200 mg every 12 hours for about 5 to 7 days that is administered orally or intravenously. In a preferred embodiment, a therapeutically effective amount of Doxycycline is a dosage of about 100 mg every 12 hours for about 5 to 7 days that is administered orally or intravenously.

Remdesivir is a nucleotide analogue prodrug that perturbs viral replication, originally evaluated in clinical trials to address the Ebola outbreak. Subsequent evaluation by numerous virology laboratories demonstrated the ability of Remdesivir to inhibit coronavirus replication, including SARS-CoV-2. Remdesivir antiviral activity, involving the interaction with the viral RdRp to induce delayed chain termination, has been demonstrated in vitro against multiple coronaviruses (SARS, MERS, contemporary human CoV and bat-CoVs). Remdesivir was also shown to perturb pan-CoV RdRp function by inhibiting viral replication of SARS, MERS, and the model β-coronavirus Murine Hepatitis Virus (MHV), including with intact exonuclease proofreading activity. Biochemical data from Recombinant Respiratory Syncytial Virus (RSV) RdRp suggested that Remdesivir's primary mechanism of action may be through delayed chain termination. Notably, Remdesivir may inhibit viral replication (as demonstrated with Ebola and RSV) in cell-based assays with IC50 values of approximately 100 nM, whereas human RNA Polymerase (RNAP) II and approximately 500-fold selectivity. This selectivity is achieved, at least in part, due to the nucleoside analogues being poor substrates for the human polymerases. Meanwhile, in vitro assays demonstrate that the triphosphate form of the inhibitor was incorporated at increased rates as compared to natural nucleotide pools, thus, Remdesivir appears to be adding strong antiviral potency through premature RNA synthesis termination.

In yet another embodiment of the present disclosure, a therapeutically effective amount of Remdesivir for slowing down the virus replication of SARS-CoV-2 is a dosage administered intravenously comprising about 200 mg on the first day, followed by about 100 mg for days 2 to 5. As can be appreciated, Remdesivir is available as a lyophilized powder and/or concentrated solution. In one embodiment, the recommended dose for adult and pediatric patients weighing 40 kg and higher may be a single loading dose of 200 mg on Day 1 followed by once daily maintenance doses of 100 mg from Day 2 onwards.

Meanwhile, for pediatric patients weighing 3.5 kg to less than 40 kg, Remdesivir may be administered intravenously with 100 mg of lyophilized powder. The recommended dose for pediatric patients weighing 3.5 kg to less than 40 kg may be a single loading dose of Remdesivir 5 mg/kg on Day 1 followed by 2.5 mg/kg once daily from Day 2 to 5. For further information on dosage, see subsection 2.3 Recommended Dosage in Pediatric Patients of the Fact Sheet for Healthcare Providers Emergency Use Authorization (EUA) of Veklury® (remdesivir) EUA Prescribing Information, as disclosed in the Information Disclosure Statement (IDS) and incorporated herein by reference in its entirety.

The optimal duration of treatment for COVID-19 varies depending on severity of the infection. In one embodiment, for patients requiring invasive mechanical ventilation and/or extracorporeal membrane oxygenation (ECMO), the recommended total treatment duration is about 10 days. Meanwhile, for patients not requiring invasive mechanical ventilation and/or ECMO, the recommended total treatment duration is about 5 days. If a patient does not demonstrate clinical improvement, treatment may be extended for up to 5 additional days for a total treatment duration of up to 10 days. As can be appreciated, Remdesivir may be administered via intravenous infusion over 30 to 120 minutes. Depending on the patient's clinical condition, these dosages can be repeated if the patient tests positive after an initial course of therapy or recurring fever after treatment.

REMDESIVIR DOSING TABLE

| | Recommended | Loading Dose on | Maintenance |
|---|---|---|---|
| Body Weight | Dosage Form | Day 1 | Dose from Day 2 |
| 3.5 Kg to less | Remdesivir | 5 mg/kg | 2.5 mg/kg |
| than 40 Kg | lyophilized | | |
| | powder for | | |
| | injection | | |
| 40 Kg | Remdesivir | 200 mg | 100 mg |
| and higher | lyophilized | | |
| | powder for | | |
| | injection | | |

Controlling the Dysregulation of Host Immune Defense:

As can be appreciated, a second component of the combination therapy is directed to regulating the patient's immune response that creates the cytokine storm, which is responsible to a greater extent for the mortality of the COVID-19. This may include administering a therapeutically effective amount of anti-inflammatory and or immune suppressant drugs like hydroxychloroquine, chloroquine, and/or steroids.

Corticosteroids (also known as glucocorticoids, cortisone or steroids) are endogenous hormones produced in the adrenal cortex or their synthetic analogues. Glucocorticoids enter cells and bind to intracellular receptors. Hydrolysis of an attached heat-shock protein releases the activated steroid-receptor complex, which is transported into the nucleus, where it binds to DNA and modifies DNA glucocorticoid-responsive elements and transcription factors. The modifications alter transcription of target genes in DNA into messenger RNA. The mRNA then leaves the nucleus and directs the synthesis of new proteins on cytoplasmic ribosomes, which are then released from the cell and elicit biological responses. Glucocorticoids in low and high doses increase the production of anti-inflammatory compounds, such as annexin-1, SLP1, MOP-1, IκB-α, GILZ, and nitric oxide synthase, and in high doses reduce the production of pro-inflammatory compounds, including cytokines, chemokines, adhesion molecules, and pro-inflammatory enzymes, such as phospholipase A2 and cyclooxygenase. The immunosuppression arises partly from inhibition of kinases responsible for cytokine production and partly from inhibition of the nuclear transcription factor NF-κB, which stimulates the transcription of cytokines, chemokines, and other molecules in the inflammatory pathway. In sum, they lessen the immune system's response.

In one embodiment of the present disclosure, a therapeutically effective dose of steroids may include oral prednisone of about 20 mg-80 mg in a tapering dose over a period of about 7 days. As can be appreciated, a therapeutically effective dose of steroids may include a dosage administered intravenously of dexamethasone (6 mg daily), or Hydrocortisone (50 mg every 8 hours daily) for about 7-14 days. These dosages may be repeated if the patient tests positive after an initial course of therapy or recurring fever after treatment. A person skilled in the art would appreciate that other steroids may also be used, such as, for example, solumedrol, to control the dysregulation of host immune defense.

Meanwhile, chloroquine/hydroxychloroquine, in its unprotonated form, can easily diffuse across cell membranes to acidic vesicles in the cytoplasm (i.e., lysosomes, late endosomes, trans-Golgi network (TGN) vesicles). Chloroquine/hydroxychloroquine may then become trapped in the vesicles after being protonated. Protonated chloroquine/hydroxychloroquine may not be able to diffuse out of lysosome or endosomes because it is retained in the cellular compartments with hydrolases. Notably, chloroquine and its analogues are known as lysosomotropic agents because they are diprotic weak bases, and their unprotonated form can selectively enter lysosomes and become protonated in a manner inversely proportional to pH (according to the Henderson-Hasselbalch law). Chloroquine/hydroxychloroquine may cause a sporadic obligation between Toll-like receptor 7 (TLR7) and TLR9 and their RNA/DNA ligands due to changing of pH towards the basic in the cellular endosomal environment, thereby suppressing TLR signaling. The impending cGAS-STING (stimulator of interferon genes) pathway causes an attenuation of pro-inflammatory cytokines, such as tumor necrosis factor (TNF), interleukin-6 (IL-6) and interleukin-1 (IL-1).

Meanwhile, enveloped viruses are trapped within the endoplasmic and TGN vesicles for post-translational modification of their envelope glycoproteins. This process involves proteases and glycosyltransferases, some of which require a low-pH environment. By neutralizing the acidic pH, chloroquine/hydroxychloroquine may deactivate several enzymes in the vesicles, such as glycosyltransferases, which in turn may be responsible for the inhibition of glycosylation. Such inhibition of glycosylation results in the host developing an adaptive immune response against the infection and impairs the cellular receptor angiotensin-converting enzyme 2 (ACE2) for SARS-CoV-1 binding and blocks fusion with the host cell. As can be appreciated, SARS-CoV-2 may also employ the ACE2 receptor to enter the host cell. In Colson P, Rolain J M, Lagier J C, Brouqui P, Raoult D. Chloroquine and hydroxychloroquine as available weapons to fight COVID-19, Int J Antimicrob Agents. 2020; 55 doi: 10.1016/j.ijantimicag.2020.105932, the author reported that the spike (S) protein of SARS-CoV-2 is cleaved in the autophagosome by host cell proteases such as cathepsins, which can be inhibited due to the increased pH in the lysosome as a result of chloroquine accumulation. Thus, chloroquine may interfere with effective binding of the spike protein to the host cell by reducing glycosylation of ACE2.

In another embodiment of the present disclosure, a therapeutically effective amount of immunosuppressant medications may include administering hydroxychloroquine (PLAQUENIL®) with an oral dosage of about 200 mg to about 400 mg twice a day for a period of about 5 to 7 days. Patients who take this medication should be monitored for prolonged QTc interval.

Preventing Coagulation and Blood Clot Formation

COVID-19 interacts with hemoglobin molecule, through CD147, CD26 and other receptors located on erythrocyte and/or blood cell precursors. Moreover, hepcidin-mimetic action of its viral spike protein may induce ferroportin blockage. Further, other pathologic metabolic pathways, derived from hemoglobin denaturation and iron metabolism dysregulation, include: a) a decrease of functioning hemoglobin quote; b) an iron overload in cell/tissue (hyperferritinemia); c) a release of free toxic circulating heme; d) a hypoxemia and systemic hypoxia; e) a reduction of nitric oxide; f) a coagulation activation; g) ferroptosis with oxidative stress and lipoperoxidation; and h) a mitochondrial degeneration and apoptosis, among others. These metabolic pathways, in addition to the cytokine storm, may or will lead to increased blood viscosity and clot formation. Therefore, COVID-19 patients may suffer stroke, pulmonary embolisms (late hypoxemia more serious), cardiovascular events, and/or multi-system failure, which leads to a high mortality rate.

As can be appreciated, a third component of the combination therapy is directed to preventing coagulation and blood clot formation. This may include administering a therapeutically effective amount of one or more of the following drugs: Acetylsalicylic acid (Asprin®), Rivaroxaban (Xeralto®), Clopidogrel (Plavix®), Enoxaparin Sodium (Lovenox®), generic formulations thereof or known substitutes thereof. These may be administered to the patient provided that the patient is not taking any other blood thinners, nor has a bleeding disorder or condition.

In one embodiment, a therapeutically effective amount of Aspirin® is a dosage of about 81-325 mg daily for a period of about 30-60 days. In a preferred embodiment, a therapeutically effective amount of Aspirin® is a dosage of about 81 mg daily for a period of about 30 days. Aspirin® [Acetylsalicylic acid (ASA)] blocks prostaglandin synthesis. It is non-selective for COX-1 and COX-2 enzymes. Inhibition of COX-1 results in the inhibition of platelet aggregation for about 7-10 days (average platelet lifespan).

In another embodiment, a therapeutically effective amount of Rivaroxaban (Xeralto®) is a dosage of about 10-20 mg/day for a period of about 30-60 days, and preferably, a dosage of about 20 mg/day for a period of about 30 days. Rivaroxaban competitively inhibits free and clot bound factor Xa. Factor Xa is needed to activate prothrombin (factor II) to thrombin (factor IIa). Thrombin is a serine protease that is required to activate fibrinogen to fibrin, which is the loose meshwork that completes the clotting process. In a further embodiment, a therapeutically effective amount of Clopidogrel (Plavix®) is a dosage of about 75 mg/day for about 30 days. In another embodiment, a therapeutically effective amount of Clopidogrel (Plavix®) is an initial dosage of about 300 mg oral loading, followed by about 75 mg/day for about 30 days. Plavix® inhibits platelet activation and aggregation through the irreversible binding of its active metabolite to the P2Y12 class of ADP receptors on platelets. In yet another embodiment, a therapeutically effective amount of Enoxaparin Sodium (Lovenox®) is a dosage of about 40 mg SQ/day for about 14 days. In severe cases, Lovenox® may be used at a dosage of about 1-1.5 mg/kg twice a day.

Vitamins and Zinc

As can be appreciated, the novel and inventive methods for the treatment of severe COVID-19 cases comprises the three components of the combination therapy. In one embodiment, the combination therapy further includes administering a therapeutically effective amount of Vitamin C, Vitamin D3 and/or Zinc. Preferably, Vitamin C, Vitamin D3 and/or Zinc are administered to all symptomatic and asymptomatic patients, as well as for preventative measures to prevent or reduce the likelihood of contracting the virus. The combination of Vitamin C, Vitamin D3 and Zinc increases the host's hostility to the virus.

In one embodiment, a therapeutically effective amount of Vitamin C is a dosage of about 1000 mg twice a day. Vitamin C is known to reduce inflammation and supports the immune system. The antiviral activities of ascorbic acid are well known. It may also support the immune system's overreaction to Covid-19. In one embodiment, a therapeutically effective amount of Vitamin D3 is a dosage of about 4000 units to about 5000 units daily. Along with promoting good bone health, Vitamin D3 may also help with immune cells function. In yet another embodiment, a therapeutically effective amount of Zinc is a dosage of about 50 mg twice a day. Zinc helps proteins throughout the body function, and people with zinc deficiencies are at higher risk of getting sick with infectious diseases.

Finally, the issues of who should be treated remains unresolved in the medical community. The answer to that is key to overcoming the COVID-19 pandemic. As can be appreciated, any patient who tested positive should be treated, regardless of whether he/she is symptomatic or not. Such protocol for COVID-19 is consistent with the strategy employed in treating tuberculosis. If the patient is symptomatic, a person of ordinary skill would understand that higher dosing or repeated course of therapy may be needed. Early treatment has very successful outcome. For example, treatment should be initiated in symptomatic patients with symptoms that are specific to COVID-19 like losing sense of taste, smell or having fever cough, or diarrhea after being exposed to a COVID patient. Of course, these patients should be tested, but therapy should be initiated regardless, even with today's high rate of false negative testing.

Persons skilled in the art would readily understand that all medications, minerals and vitamins can be adjusted to their maximum therapeutic tolerated dose. It is understood that the disclosure is not limited to the particular methodology, protocols, and dosages described as these may vary. It is also to be understood that the terminology used herein is for the purpose of describing particular embodiments only, and is not intended to limit the scope of the present invention which will be limited only by the appended claims.

Although the various inventive aspects are herein disclosed in the context of certain preferred embodiments, implementations, and examples, it will be understood by those skilled in the art that the present invention extends beyond the specifically disclosed embodiments to other alternative embodiments and/or uses of the invention and obvious modifications and equivalents thereof. In addition, while a number of variations of the inventive aspects have been shown and described in detail, other modifications, which are within their scope will be readily apparent to those of skill in the art based upon this disclosure. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. Thus, it is intended that the scope of the present invention herein disclosed should not be limited by the particular disclosed embodiments or implementations described above, but should be determined only by a fair reading of the claims.

Similarly, this disclosure is not to be interpreted as reflecting an intention that any claim requires more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

Further, all claim terms should be interpreted in their most expansive forms so as to afford the applicant the broadest coverage legally permissible. Although the embodiments have been described with reference to specific examples, it will readily be appreciated by those skilled in the art that many modifications and adaptations of the processes, methods and apparatuses described herein are possible without departure from the spirit and scope of the embodiments as claimed herein. Thus, it is to be clearly understood that this description is made only by way of example and not as a limitation on the scope of the embodiments as claimed below.

1. A method for treating a COVID-19 infection, the method comprising:
administering a therapeutically effective amount of a combination of at least four components concurrently,
a first component is selected from a group consisting of azithromycin, doxycycline, and remdesivir,
a second component is selected from a group consisting of hydroxychloroquine and a steroid,
a third component is selected from a group consisting of acetylsalicylic acid, rivaroxaban, clopidogrel, and enoxaparin sodium, and
a fourth component is a therapeutically effective amount of Vitamin C, Vitamin D3 and Zinc.

2. (canceled)

3. The method of claim 1, wherein the therapeutically effective amount of azithromycin is a dosage of about 250 to 500 mg/day for a period of about 5 to 7 days.

4. The method of claim 1, wherein the therapeutically effective amount of doxycycline is a dosage of about 100 mg every 12 hours for about 5 days.

5. The method of claim 1, wherein the therapeutically effective amount of remdesivir is a dosage administered intravenously comprising about 200 mg on the first day, followed by about 100 mg for days 2 to 5.

6. The method of claim 1, wherein the steroid is selected from a group consisting of prednisone, hydrocortisone, solumedrol and dexamethasone.

7. The method of claim 6, wherein the therapeutically effective amount of prednisone is a dosage of about 20 mg-80 mg in a tapering dose over a period of about 7 days, the therapeutically effective amount of hydrocortisone is a dosage of about 50 mg every 8 hours daily for about 7-14 days, and the therapeutically effective amount of dexamethasone is a dosage administered intravenously comprising about 6 mg daily.

8. The method of claim 1, wherein the therapeutically effective amount of hydroxychloroquine is a dosage of about 200 to about 400 mg/day for a period of about 5 to 7 days.

9. The method of claim 1, wherein the therapeutically effective amount of acetylsalicylic acid is a dosage of about 81 mg to about 325 mg daily for a period of about 30 to 60 days.

10. The method of claim 1, wherein the therapeutically effective amount of rivaroxaban is a dosage of about 10 mg/day to about 20 mg/day for a period of about 30 days to about 60 days.

11. The method of claim 1, wherein the therapeutically effective amount of clopidogrel is a dosage of about 75 mg/day for about 30 days.

12. The method of claim 1, wherein the therapeutically effective amount of enoxaparin sodium is a dosage of about 40 mg SQ/day for about 14 days.

13. The method of claim 1, wherein the therapeutically effective amount of Vitamin C is a dosage of about 1000 mg twice a day, the therapeutically effective amount of Vitamin D3 is a dosage of about 2000 units twice a day, and the therapeutically effective amount of Zinc is a dosage of about 50 mg twice a day.

14. A method of reducing the effect of a Severe Acute Respiratory Syndrome Coronavirus 2 (SARS-CoV-2) in a patient, the method comprising:
administering to the patient a therapeutically effective amount of a combination of three components consisting essentially of:
a first component selected from a group consisting of azithromycin, doxycycline, and remdesivir, the therapeutically effective amount of azithromycin is a dosage of about 250 to 500 mg/day for a period of about 5 to 7 days, the therapeutically effective amount of doxycycline is a dosage of about 50 mg to about 200 mg every 12 hours for about 5 days, and the therapeutically effective amount of remdesivir is a dosage administered intravenously comprising about 200 mg on the first day, followed by about 100 mg for days 2 to 5,
a second corticosteroid component selected from a group consisting of prednisone and hydrocortisone, the therapeutically effective amount of prednisone is an oral dosage of about 20 mg-80 mg in a tapering dose over a period of about 7 days, and the therapeutically effective amount of hydrocortisone is an oral dosage of about 50 mg every 8 hours, and
a third component is selected from a group consisting of acetylsalicylic acid, rivaroxaban, clopidogrel, and enoxaparin sodium.

15. (canceled)

16. (canceled)

17. The method of claim 14, wherein the therapeutically effective amount of acetylsalicylic acid is a dosage of about 81 mg daily for a period of about 30 days, the therapeutically effective amount of rivaroxaban is a dosage of about 20 mg/day for a period of about 30 day, the therapeutically effective amount of clopidogrel is a dosage of about 75 mg/day for about 30 days and the therapeutically effective amount of enoxaparin sodium is a dosage of about 40 mg SQ/day for about 14 days.

18. The method of claim 14, further comprising administering a therapeutically effective amount of Vitamin C, Vitamin D3 and Zinc, the therapeutically effective amount of Vitamin C is a dosage of about 1000 mg twice a day, the therapeutically effective amount of Vitamin D3 is a dosage of about 2000 units twice a day, and the therapeutically effective amount of Zinc is a dosage of about 50 mg twice a day.

19. A method of treating a COVID-19 infection in a patient, the method comprising:
concurrently administering to the patient a therapeutically effective amount of a combination of four components consisting essentially of:
a first component for slowing down viral replication, the first component is selected from a group consisting of azithromycin, doxycycline, and remdesivir, the therapeutically effective amount of azithromycin is a dosage of about 250 to 500 mg/day for a period of about 5 to 7 days, the therapeutically effective amount of doxycycline is a dosage of about 100 mg every 12 hours for about 5 days, and the therapeutically effective amount of remdesivir is a dosage administered intravenously comprising about 200 mg on the first day, followed by about 100 mg for days 2 to 5,
a second corticosteroid component for controlling dysregulation of the patient's immune defense, the second corticosteroid component selected from a group consisting of prednisone and hydrocortisone, the therapeutically effective amount of prednisone is an oral dosage of about 20 mg-80 mg in a tapering dose over a period of about 7 days, and the therapeutically effective amount of hydrocortisone is an oral dosage of about 50 mg every 8 hours,
a third component for preventing coagulation and blood clot formation, the third component selected from a group consisting of acetylsalicylic acid, clopidogrel, and enoxaparin sodium, the therapeutically effective amount of acetylsalicylic acid is a dosage of about 81 mg daily for a period of about 30 days, the therapeutically effective amount of clopidogrel is a dosage of about 75 mg/day for about 30 days and the therapeutically effective amount of enoxaparin sodium is a dosage of about 40 mg SQ/day for about 14 days, and
a fourth component for supporting the patient's immune system, the fourth component including Vitamin C, Vitamin D3 and Zinc, the therapeutically effective amount of Vitamin C is a dosage of about 1000 mg twice a day, the therapeutically effective amount of Vitamin D3 is a dosage of about 2000 units twice a day, and the therapeutically effective amount of Zinc is a dosage of about 50 mg twice a day.

20. (canceled)
